(19) United States
(12) Patent Application Publication
(10) Pub. No.: US 2009/0188500 A1
(43) Pub. Date: Jul. 30, 2009

(54) COMBINATION BREATHING TREATMENT METHOD

(76) Inventor: Joseph Dee FARAM, Dallas, TX (US)

Correspondence Address: HITCHCOCK EVERT LLP, P.O. BOX 131709, DALLAS, TX, 75313-1709, US

(21) Appl. No.: 12/361,914

(22) Filed: Jan. 29, 2009

Related U.S. Application Data
(60) Provisional application No. 61/024,544, filed on Jan. 29, 2008.

(51) Int. Cl. A61M 16/00

(52) U.S. Cl. 128/204.18

(57) ABSTRACT

A method of providing a combination lung treatment therapy that is substantially uninterrupted between providing continuous positive airway pressure therapy and high-frequency therapy is disclosed. The present invention combines multiple respiratory therapies in order to facilitate the prevention and treatment of certain pulmonary diseases. The present invention also discloses a method of providing a combination lung treatment therapy that is substantially uninterrupted between providing medicated continuous positive airway pressure therapy and medicated high-frequency therapy is disclosed.

### CROSS-REFERENCE TO RELATED APPLICATION

This application claims the benefit of priority from U.S. Provisional Application No. 61/024,544 filed on Jan. 29, 2008.

### FIELD OF THE INVENTION

The invention relates to a breathing treatment that combines multiple respiratory therapies in order to facilitate the prevention and treatment of certain pulmonary diseases.

### BACKGROUND OF THE INVENTION

Atelectasis is the partial or total collapse of the lung. Although this condition may occur as a result of pressure being exerted from outside the lungs by such maladies as a tumor or fluid buildup in the pleural space, it is most often caused by obstruction within the airways. When a blockage develops, the air in the small air sacs, or alveoli, on the distal side of the obstruction is absorbed into the bloodstream. The air sacs thus become diminished in size and/or collapse. The alveoli then may fill with blood cells, mucus, or serum, making them highly susceptible to infection. Atelectasis may happen suddenly or gradually manifest over a long period of time. In either case the disorder may lead to shortness of breath, decreased oxygen levels in the bloodstream, increased heart rate, and infection resulting in outcomes ranging from simple discomfort to death.

Three modes of treatment have each been found to be beneficial in the prevention and treatment of atelectasis: 1) inhaled medicated aerosol, 2) Continuous Positive Airway Pressure, or “CPAP”, for lung expansion therapy, and 3) high-frequency positive airway pressure for secretion clearance therapy. It is believed that high-frequency therapy is enhanced by simultaneously delivering medicated aerosol. Likewise, CPAP is enhanced by combining it with aerosol therapy. However, a problem arises when one of these two combination therapies is delivered.

When aerosol is combined with high-frequency therapy the turbulent flow of gas significantly diminishes the respirable quantity of medicated aerosol delivered. However, when aerosol is combined with CPAP therapy the respirable quantity of medicated aerosol is significantly enhanced, but the CPAP-aerosol therapy alone may well be insufficient in mobilizing secretions in the airways.

While the respective benefits of these two types of therapies have been known, various obstacles have prevented their combined use. For example, in some applications, it has been difficult or impossible to switch a patient between CPAP and high frequency therapy in an uninterrupted manner. Specifically, some applications have required the use of multiple devices to achieve the multiple types of therapy. Thus, when switching between therapies and corresponding apparatus, it has been necessary to momentarily halt therapy.

Thus, there has been a need for a method which combines the benefits of medicated CPAP therapy with medicated high frequency therapy, creating a combined breathing treatment therapy.

### SUMMARY OF THE INVENTION

The present invention combines aerosol medication delivery, CPAP lung expansion therapy, and high-frequency secretion mobilization therapy into a single breathing treatment. Aerosolized medication delivery is combined with a high frequency pulsatile gas flow to provide a secretion mobilization mode, and with a CPAP flow to provide a lung expansion mode. The invention is comprised of the method of providing uninterrupted therapy while alternating between pulsatile secretion clearance mode and CPAP lung expansion mode.

Thus, an object of the present invention is to provide a method of uninterrupted respiratory therapy combining the benefits of medicated CPAP therapy with medicated high frequency therapy.

A further object of the present invention is to provide a method of uninterrupted respiratory therapy combining the benefits of medicated CPAP therapy with medicated high frequency therapy wherein the periods of application of each therapy are fixed.

A further object of the present invention is to provide a method of uninterrupted respiratory therapy combining the benefits of medicated CPAP therapy with medicated high frequency therapy wherein the therapy consists of alternating between medicated CPAP therapy and medicated high frequency therapy.

A further object of the present invention is to provide a method of uninterrupted respiratory therapy combining the benefits of medicated CPAP therapy with medicated high frequency therapy wherein the total time period of therapy is divided equally between medicated CPAP therapy and medicated high frequency therapy.

A further object of the present invention is to provide a method of uninterrupted respiratory therapy combining the benefits of medicated CPAP therapy with medicated high frequency therapy wherein medicated CPAP therapy is given for greater time than medicated high frequency therapy.

A further object of the present invention is to provide a method of uninterrupted respiratory therapy combining the benefits of medicated CPAP therapy with medicated high frequency therapy wherein medicated high frequency therapy is given for greater time than medicated CPAP therapy.

### DETAILED DESCRIPTION

In the method of the present invention, a patient is administered a combination of both medicated CPAP therapy as well as medicated high frequency therapy. By exposing a patient to both forms of therapy, the patient receives the benefits of both types of therapies, while avoiding the negative effects typical to each therapy. In the method of the present invention, a patient is placed in fluid communication with a breathing treatment apparatus of a type commonly known in the art. The breathing treatment apparatus is placed in fluid communication with a source of gas, such as a flow meter that regulates the flow of air or oxygen from a wall outlet. Therapy is most often delivered to the patient through a mouthpiece or mask.

In one embodiment, treatment may begin by administering a first mode of therapy, either medicated CPAP therapy or high frequency therapy. Following a period of time in which the first therapy is administered, the first therapy may be discontinued and the second mode of therapy, the second mode being whichever mode was not administered as the first mode of therapy, begun without substantial interruption of the treatment. That is, treatment is substantially uninterrupted while the switch is made from the first mode of therapy to the second mode of therapy.

In a further embodiment of the present invention, the cumulative time in which the first mode of therapy and the second mode of therapy are administered during a single treatment session are approximately equal.

In a further embodiment of the present invention, each mode of therapy is administered for a predetermined amount of time, set prior to commencement of the therapy session.

Although the invention has been disclosed and described in relation to its preferred embodiments with a certain degree of particularity, it is understood that the present disclosure of some preferred forms is only by way of example and that numerous changes in the details of construction and operation and in the combination and arrangements of parts may be resorted to without departing from the spirit of the scope of the invention as claimed here.

What is claimed:

1. A method of administering a combination lung therapy comprising the steps of:
placing a patient in fluid communication with a breathing treatment apparatus;
placing said breathing treatment apparatus in fluid connection with a source of gas;
administering continuous positive airway pressure therapy to the patient via said breathing treatment apparatus;
discontinuing continuous positive airway pressure therapy to the patient;
administering high-frequency therapy to the patient via said breathing treatment apparatus; and
discontinuing continuous high-frequency therapy to the patient.

2. The method of administering a combination lung therapy according to claim 1 further comprising the steps of:
filling a small-volume nebulizer with an appropriate amount and kind of medication; and
connecting said small-volume nebulizer to said breathing treatment apparatus prior to administering continuous positive airway pressure therapy to the patient.

3. The method of administering a combination lung therapy according to claim 1 further comprising the steps of:
filling a small-volume nebulizer with an appropriate amount and kind of medication; and
connecting said small-volume nebulizer to said breathing treatment apparatus prior to administering high-frequency therapy to the patient.

4. The method of administering a combination lung therapy according to claim 1 wherein the step of administering continuous positive airway pressure therapy to the patient via said breathing treatment apparatus takes the same amount of time as the step of administering high-frequency therapy to the patient via said breathing treatment apparatus.

5. The method of administering a combination lung therapy according to claim 1 wherein the step of administering continuous positive airway pressure therapy to the patient via said breathing treatment apparatus takes a greater amount of time than the step of administering high-frequency therapy to the patient via said breathing treatment apparatus.

6. The method of administering a combination lung therapy according to claim 1 wherein the step of administering continuous positive airway pressure therapy to the patient via said breathing treatment apparatus takes less time than the step of administering high-frequency therapy to the patient via said breathing treatment apparatus.

7. The method of administering a combination lung therapy according to claim 1 further comprising the steps of:
repeating the following steps throughout the combination lung therapy:
administering continuous positive airway pressure therapy to the patient via said breathing treatment apparatus;
discontinuing continuous positive airway pressure therapy to the patient;
administering high-frequency therapy to the patient via said breathing treatment apparatus; and
discontinuing continuous high-frequency therapy to the patient.

8. The method of administering a combination lung therapy according to claim 7 further comprising the steps of:
filling a small-volume nebulizer with an appropriate amount and kind of medication; and
connecting said small-volume nebulizer to said breathing treatment apparatus prior to administering continuous positive airway pressure therapy to the patient.

9. The method of administering a combination lung therapy according to claim 7 further comprising the steps of:
filling a small-volume nebulizer with an appropriate amount and kind of medication; and
connecting said small-volume nebulizer to said breathing treatment apparatus prior to administering high-frequency therapy to the patient.

10. The method of administering a combination lung therapy according to claim 7 wherein the step of administering continuous positive airway pressure therapy to the patient via said breathing treatment apparatus takes the same amount of time as the step of administering high-frequency therapy to the patient via said breathing treatment apparatus.

11. The method of administering a combination lung therapy according to claim 7 wherein the step of administering continuous positive airway pressure therapy to the patient via said breathing treatment apparatus takes a greater amount of time than the step of administering high-frequency therapy to the patient via said breathing treatment apparatus.

12. The method of administering a combination lung therapy according to claim 7 wherein the step of administering continuous positive airway pressure therapy to the patient via said breathing treatment apparatus takes less time than the step of administering high-frequency therapy to the patient via said breathing treatment apparatus.

13. A method of administering a combination lung therapy comprising the steps of:
placing a patient in fluid communication with a breathing treatment apparatus;
placing said breathing treatment apparatus in fluid connection with a source of gas;
administering high-frequency therapy to the patient via said breathing treatment apparatus;
discontinuing continuous high-frequency therapy to the patient;
administering continuous positive airway pressure therapy to the patient via said breathing treatment apparatus; and
discontinuing continuous positive airway pressure therapy to the patient.

14. The method of administering a combination lung therapy according to claim 13 further comprising the steps of:
filling a small-volume nebulizer with an appropriate amount and kind of medication; and
connecting said small-volume nebulizer to said breathing treatment apparatus prior to administering continuous positive airway pressure therapy to the patient.

15. The method of administering a combination lung therapy according to claim 13 further comprising the steps of:
filling a small-volume nebulizer with an appropriate amount and kind of medication; and
connecting said small-volume nebulizer to said breathing treatment apparatus prior to administering high-frequency therapy to the patient.

16. The method of administering a combination lung therapy according to claim 13 wherein the step of administering continuous positive airway pressure therapy to the patient via said breathing treatment apparatus takes the same amount of time as the step of administering high-frequency therapy to the patient via said breathing treatment apparatus.

17. The method of administering a combination lung therapy according to claim 13 wherein the step of administering continuous positive airway pressure therapy to the patient via said breathing treatment apparatus takes a greater amount of time than the step of administering high-frequency therapy to the patient via said breathing treatment apparatus.

18. The method of administering a combination lung therapy according to claim 13 wherein the step of administering continuous positive airway pressure therapy to the patient via said breathing treatment apparatus takes less time than the step of administering high-frequency therapy to the patient via said breathing treatment apparatus.

19. The method of administering a combination lung therapy according to claim 13 further comprising the steps of:
repeating the following steps throughout the combination lung therapy:
administering high-frequency therapy to the patient via said breathing treatment apparatus;
discontinuing continuous high-frequency therapy to the patient;
administering continuous positive airway pressure therapy to the patient via said breathing treatment apparatus; and
discontinuing continuous positive airway pressure therapy to the patient.

20. The method of administering a combination lung therapy according to claim 19 further comprising the steps of:
filling a small-volume nebulizer with an appropriate amount and kind of medication; and
connecting said small-volume nebulizer to said breathing treatment apparatus prior to administering continuous positive airway pressure therapy to the patient.

21. The method of administering a combination lung therapy according to claim 19 further comprising the steps of:
filling a small-volume nebulizer with an appropriate amount and kind of medication; and
connecting said small-volume nebulizer to said breathing treatment apparatus prior to administering high-frequency therapy to the patient.

22. The method of administering a combination lung therapy according to claim 19 wherein the step of administering continuous positive airway pressure therapy to the patient via said breathing treatment apparatus takes the same amount of time as the step of administering high-frequency therapy to the patient via said breathing treatment apparatus.

23. The method of administering a combination lung therapy according to claim 19 wherein the step of administering continuous positive airway pressure therapy to the patient via said breathing treatment apparatus takes a greater amount of time than the step of administering high-frequency therapy to the patient via said breathing treatment apparatus.

24. The method of administering a combination lung therapy according to claim 19 wherein the step of administering continuous positive airway pressure therapy to the patient via said breathing treatment apparatus takes less time than the step of administering high-frequency therapy to the patient via said breathing treatment apparatus.
